(19) United States
(12) Patent Application Publication
(10) Pub. No.: US 2009/0054741 A1
(43) Pub. Date: Feb. 26, 2009

(54) Device and method of monitoring a patient

(75) Inventor: Jerry McAleer, Oxfordshire (GB)

Correspondence Address: STEPTOE & JOHNSON LLP, 1330 CONNECTICUT AVENUE, N.W., WASHINGTON, DC, 20036, US

(73) Assignee: Inverness Medical Switzerland GMBH, Zug (CH)

(21) Appl. No.: 11/887,139

(22) PCT Filed: Mar. 24, 2006
(86) PCT No.: PCT/IB2006/001776

§ 371 Date: Oct. 30, 2008

Related U.S. Application Data
(60) Provisional application No. 60/665,864, filed on Mar. 29, 2005.

(51) Int. Cl. A61B 5/00

(52) U.S. Cl. 600/301

(57) ABSTRACT

A device for remote management of patients suffering or likely to suffer from heart failure that can measure the amplitude and frequency changes of one or more biomarkers. The device aids in predicting the need for medical intervention in such patients. The device may further aid in monitoring the efficacy and safety of treatment in such patients.

### TECHNICAL FIELD

This invention relates to a method and apparatus for monitoring a patient for heart failure.

### BACKGROUND

Heart failure is a chronic, progressive disease that affects 1.5-2% of the general population of the Western world. The prevalence and incidence of heart failure is growing due to an aging population. Heart failure occurs when the heart is not strong enough to pump blood efficiently around the body.

Heart failure is often the result of acute cardiovascular events such as stroke or myocardial infarction (MI). These events are commonly preceded by rupture of an unstable plaque resulting in thrombus formation within a coronary blood vessel. The thrombus impedes blood flow restricting oxygen supply to the cardiac muscle resulting ultimately in cell death (necrosis). These attacks may be fatal and at the very least will impair future quality of life. The problem arises from the fact that there is generally little obvious external warning of an impending MI and even when the MI takes place, it is often difficult to diagnose until severe damage has been done. Furthermore, patients who survive an infarct often go on to have a subsequent infarct or begin to suffer from congestive heart failure.

A consequence of this is that cardiovascular disease places an ever increasing burden on healthcare. In the United States alone, there are over 5 million sufferers from Congestive Heart Failure (CHF). An ideal solution to the problem would be to intervene before MI or CHF occurs by monitoring the likelihood that an unstable plaque may rupture or by monitoring thrombus formation. Preventive measures may then be taken to prevent MI or CHF as early as possible.

Many of the tests and procedures for accurately and successfully monitoring, diagnosing, managing and treating heart failure are complex, expensive and available only at a hospital or other health-care settings. Methods for patients to manage or to monitor the likelihood of heart failure at home or otherwise outside a health-care setting are even less successful.

### SUMMARY

A patient with pre-heart failure or heart failure can be managed in the home or a non-hospital setting. To help the patient monitor the likelihood of cardiovascular events occurring or manage heart failure, a means is provided to detect or to monitor the patient's condition. Cardiovascular events may include but are not limited to myocardial infarction, stroke, unstable angina, cardiac thrombus, resuscitated cardiac arrest, sudden or unexplained death, ischemic stroke, and transient ischemic attack. Such a device is also useful for patients at risk of further myocardial infarctions, for example, a patient who has survived a first myocardial infarction and is at risk for future myocardial infarction.

The device can detect or monitor predictive factors in patients that may be considered “at risk” patients. The device may be used to monitor predictive factors in any patient. Predictive factors are not the same as risk factors. Well established risk factors are, for example, smoking, obesity, diabetes, and hypertension. These provide a general measure of risk but have no real predictive value within an individual, only in a general sense across populations. Given the limitations of such risk factors, the current option to predict heart failure is the determination of cardiac markers post-infarction.

The device is based on monitoring predictive factors which are biomarkers that can be measured and trended within an individual to provide advance warning of an event, in the same way that oil temperature and pressure gauges give an indication of engine well-being. Such biomarkers may include a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention. The levels of such biomarkers may from time to time experience “tremors” which are excursions from baseline levels (i.e. increased frequency and amplitude of measured biomarker levels). The device can be used to track such excursions and measure the frequency and amplitude of these excursions. The device may further be used to track biomarkers that are correlated and predict the likelihood of a patient experiencing a cardiovascular event. For example, a peak in a marker of inflammation such as C-reactive protein, followed a day later by a peak in a marker of macrophage activity such as myeloperoxidase (MPO) followed by a peak in a marker for plaque instability such as oxidized-LDL will signal that an atherosclerotic plaque rupture is imminent. Such correlated peaks may appear rarely but as heart failure progresses, these peaks may appear more frequently until just prior to the catastrophic event when all markers of heart failure erupt to higher levels.

The device can detect or monitor, for example, indications of plaque instability, episodes of plaque rupture, episodes of thrombus formation, episodes of myocardial ischemia, episodes of myocardial apoptosis or infarction, onset of acute decompensation, episodes of acute decompensation, episodes of hypoxia, response to diuretic therapy, response to fluid intake, response to sodium intake, response to primary pharmacological agents (e.g., ACE inhibitor, β-blocker, aldosterone II receptor antagonist), and response to secondary pharmacological agents (e.g., hydralazine/isosorbide dinitrate).

The device can also be used to track certain predictive factors in “high risk” individuals (perhaps selected on the basis of conventional risk factors) and provide advance warning of an impending cardiovascular event. The device may also be used in an acute care setting to allow early intervention.

The device allows the patient to perform serial measurements of one or more biomarkers at regular intervals, collect information on signs and symptoms by paper chart or electronic diary, detect concentration excursions from average concentration levels, detect the frequency of such concentration excursions and, if necessary, to compute such measurements of biomarker(s) with other parameters such as signs and symptoms (e.g. breathlessness, cough, edema, decreased exercise tolerance, unexplained confusion or altered mental state, weight gain, fatigue, abdominal symptoms or signs related to ascites and hepatic engorgement, blood pressure, heart rate, variability of heart rate, and oxygen saturation). The biomarkers measured by the device can include, but are not limited to, a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

Because the test is simple enough to be carried out in the patient's home, daily measurements can be obtained and allow for an earlier notification of a detrimental change in the patient's cardiovascular condition than would otherwise be possible. Thus, the patient or a healthcare professional is able to review real-time data on the patient's likelihood for developing a cardiovascular event or the patient's pathophysiological state and response to therapy.

In one aspect, a method and device to determine the likelihood of a cardiovascular event occurring or to determine pathophysiological status and therapeutic response of a mammalian subject, includes a detector for measuring, in a sample taken from the subject, the level of biomarkers which may include a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The detector can be associated with a device for providing a display of the result of the measured parameters, and a means to manually or automatically input data from other measurements or observations or risk factors. The other measurements, observations or risk factors can including breathlessness, cough, edema, decreased exercise tolerance, unexplained confusion or altered mental state, weight gain, fatigue, abdominal symptoms or signs related to ascites and hepatic engorgement, blood pressure, heart rate, heart rate variability, oxygen saturation, age, gender, body mass index, frequency and volume of urination, dry cough, dry mouth, nausea, pain, fluid intake, salt intake, drug administration, exercise, weight control, and assessment of quality of life.

In another aspect, a method includes inputting a series of preset or predetermined levels (decision points) for each biomarker (e.g. a baseline level and a single or multiple action levels) and calculating the excursions in levels of the biomarker from the predetermined levels.

A baseline level for a marker may be assigned when the patient is stabilized or when the patient has not experienced any cardiovascular events over a period of time. The baseline can include periodic variations in marker levels that are within normal levels. The baseline level can be a normal or target level. Relative changes with respect to the baseline value which occur from increased frequency and/or at higher amplitudes will reflect deterioration or improvements in the patient's status allowing intervention by the patient or healthcare provider if necessary.

An action level for a marker is a level sufficiently separated from the baseline level that occurs at increased frequency and/or amplitude to indicate a change in the patient's condition. This would result in the patient and, if necessary, the healthcare professional being alerted to a change in status. If appropriate, a recommended course of action can be relayed via the display or another means of communication. Changes relative to the action level would indicate improvements or further deterioration in the patient's condition.

The absolute level, or the frequency of change, or the magnitude of change in the measured parameter can be compared to a predetermined level, such as a previously stored measurement or a preset action level.

The result of a measurement can be stored. The measurement can include raw data or interpreted data, such as absolute biomarker concentration, biomarker level relative to a preset action level, rate of change of the biomarker, magnitude of change of the biomarker, or any manually or automatically entered parameter. The measurement may further be compared to measurements of other correlated biomarkers.

The outcome of any measured or interpreted parameter or any manually or automatically entered parameter can be compared to the result for any other parameter.

The device can display and store in memory the findings of any of the above outcomes.

The device can relay stored data to a healthcare professional or other caregiver.

The device can be configured to determine when the user should perform a test or evaluate any other parameter.

The device can be configured to determine whether the user performed a test, administered a drug or any other intervention, or evaluated any other parameter.

The device can upload data from the instrument or to download data to the instrument.

In another aspect, the device for predicting heart failure includes a detector configured to monitor concentration excursions, in samples taken from a patient at regular intervals, a level of a first biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The device can be configured to detect the frequency of concentration excursions from average concentrations of the first biomarker. The device can include a display configured to provide an output to the patient. The detector can be configured to monitor concentration excursions of the level of a second biomarker. The detector can be configured to monitor concentration excursions of the level of a third biomarker.

The second biomarker can be a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, or a marker of sodium retention.

The third biomarker can be a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, or a marker of sodium retention.

In certain circumstances, the first biomarker can be a marker of plaque instability. In other circumstances, the first biomarker can be a marker of inflammation and the second biomarker can be a marker of plaque instability. In other circumstances, the first biomarker can be a marker of inflammation, the second biomarker can be a marker of plaque instability, and the third biomarker can be a marker of plaque rupture.

The marker of inflammation can include E-selectin, P-selectin, intracellular adhesion molecule-1, vascular cell adhesion molecule-1, Nourin-1, interleukin-1β, interleukin-6, interleukin-8, interleukin-10, tumor necrosis factor-alpha, hs-CRP, myeloperoxidase, neutrophils, or white blood cell count. The marker of plaque instability can include oxidized-LDL. The marker of thrombus formation can include (fe) thromboxane. The marker of plaque rupture can include malondialdehyde-modified LDL (MDA-LDL). The marker of myocardial apoptosis or injury can include cardiac troponin I, troponin T, myoglobin, creatine kinase or creatine kinase MB (CK MB), urotensin, or urotensin-related peptide. The marker of myocardial ischemia can include ischemia-modified albumin, oxygen-regulated peptide (ORP150), free fatty acid, Nourin-1, urotensin, or urotensin-related peptide. The marker of anemia can include hemoglobin or hematocrit. The marker of renal function can include creatinine or Cystatin C. The marker of electrolyte balance can include Na⁺ or K⁺. The marker of sodium retention can include uroguanylin.

The device can further include a probe for measuring a vital sign of the patient. The probe can measure weight, a heart rate, variability of heart rate, a breathing rate, a blood pressure, a temperature, a blood oxygen saturation, or an electrocardiogram of the patient.

The device can include a memory capable of storing the results of regular measurements of the level of the first biomarker. The device can be configured to compare the result of a measurement of the level of the first biomarker to stored results from previous measurements. The memory can store a threshold value of the level of the first biomarker.

The device can be configured to compare the result of a measurement of the level of the first biomarker to the threshold value and to previous measurements. The device can be configured to instruct the patient to contact his physician when the device detects concentration excursions in the levels of the first biomarker. The device can also be configured to instruct the patient to alter a treatment plan when the device detects concentration excursions in the levels of the first biomarker occurring at high frequency. The device can be configured to further instruct the patient to obtain a measurement of a second biomarker and/or third biomarker.

The device can include a display for displaying the results of the measurement, a patient query, or a patient instruction. The device can include an input device for supplying a response to a patient query. The device can be configured to provide a personalized patient instruction in response to the results of the measurement. The device can include a communication port configured to transmit a result of a measurement to a recipient. The communication port can be configured to receive information from the recipient.

In another aspect, a method of monitoring a patient for heart failure includes measuring and detecting concentration excursions, in samples taken from a patient at regular intervals, the levels of a first biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The method can include providing an output to the patient. The method can include comparing the measured level of the first biomarker to a threshold value and to previous measurements. The method can further include instructing the patient to obtain a measurement of a second biomarker and/or a third biomarker when rapid concentration excursions are detected in the levels of the first biomarker. The method can further include measuring and detecting concentration excursions in a sample taken from a patient, a level of a second biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The method can also include measuring and detecting concentration excursions in a sample taken from a patient, a level of a third biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention. The method can include determining whether the patient is suffering from one or more symptoms associated with heart failure. The method also includes measuring a weight, a heart rate, variability of heart rate, a breathing rate, a blood pressure, a temperature, a blood oxygen saturation, or an electrocardiogram of the patient.

In another aspect, a health care kit can include a test cartridge including a sample port and a first assay, wherein the first assay recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention; and a device including a detector configured to measure and to monitor concentration excursions of a level of the biomarker recognized by the assay. The device can be configured to provide an output to a patient. The first assay can include an antibody that recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The kit can include a second test cartridge including a sample port and a second assay, wherein the second assay recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention. The second assay can include an antibody that recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The kit can include a third test cartridge including a sample port and a third assay, wherein the second assay recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention. The third assay can include an antibody that recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

The test cartridge can include a second assay, the second assay being different from the first assay.

The details of one or more embodiments are set forth in the drawings and description below. Other features, objects, and advantages will be apparent from the description, the drawings, and from the claims.

### BRIEF DESCRIPTION OF THE DRAWING

FIG. 1 is diagram illustrating a diagnostic device and an associated testing cartridge.

### DETAILED DESCRIPTION

A patient with heart failure can be a patient at high-risk of experiencing a cardiovascular event or developing heart failure (patients with coronary heart disease, diabetes, hypertension, and/or valvular heart disease) (Stage A), pre-heart failure: patients with structural heart disease but without clinical heart failure symptoms, many of whom have decreased systolic function (Stage B), heart failure patients who have prior or current symptomatic heart failure due to systolic or diastolic dysfunction and who are responding to therapy (Stage C), and advanced heart failure patients in end-stage or refractory-to-therapy conditions (Stage D).

At risk patients (for example, patients with coronary heart disease) also suffer from a high probability of hypoxia, myocardial ischemia, and myocardial infarcts. Acute myocardial infarction (AMI) and non-haemorrhagic stroke may occur as the result of thrombus formation, which itself is the result of the rupture of an atherosclerotic plaque with subsequent clot formation. Early detection of thrombus formation would allow medical intervention such as the use of aspirin or thrombolytic agents. However, detection of the plaque forming, becoming unstable and becoming prone to rupture would be much better since medical intervention (e.g. plaque stabilization) would be possible at a much earlier stage. For example, a patient who is aware of the imminency of plaque rupture may be treated with Statins to prevent plaque rupture and sequelae. The efficacy of the medication could be monitored by tracking the same biomarkers whose levels would subside as inflammation is suppressed and the plaque cap thickens. In the event that the appropriate intervention does not take place or is ineffective, plaque rupture may be determined by, for example, an acute rise in circulating MDA-LDL levels or platelet activation markers. An intervention at this stage will may assist in preventing thrombus formation and further damage resulting from these events.

In a patient with heart failure, cardiac output is inadequate to meet the metabolic needs of the body, either at rest or with exercise. An increase in cardiac filling pressure or volume usually occurs as well. Heart failure is most commonly due to left ventricular systolic dysfunction (LVSD) where the myocardium fails to contract normally and the left ventricle is usually dilated. As the disease progresses, the body responds to the diminished cardiac output through activation of the renin-angiotensin-system (RAS) causing arterial vasoconstriction, enhanced sodium reabsorption, and volume expansion. There is an increase in presynaptic stimulation of sympathetic nerves to enhance norepinephrine release, which is deleterious in the long-term for the patient. These effects, which are mediated by angiotensin II binding to the AT₁ receptor, are immediate and are compensatory changes that develop to augment cardiac output and increase perfusion pressure to vital organs. In addition to these immediate hemodynamic effects, angiotensin II also causes cardiac remodeling through fibroblast and myocyte proliferation. Remodeling involves increases in left ventricle volume and mass, as well as changes in conformation that ultimately lead to diastolic and systolic dysfunction. Another immediate effect of angiotensin II relevant to the heart failure patient is an increased thirst caused by the release of arginine vasopressin which can exacerbate the fluid retention. Symptoms and signs of heart failure and a worsening condition include breathlessness, cough, edema in the lower extremities, decreased exercise tolerance, unexplained confusion or altered mental state, weight gain, fatigue, and abdominal symptoms or signs related to ascites and hepatic engorgement.

The overall treatment plan for a patient with hypertension, pre-heart failure (Stage B) or heart failure (Stages C or D) includes careful management of pharmacological therapy, diet and lifestyle. The primary goals are prolongation of the patient's life by preventing, slowing, halting, or reversing the progressive condition, relief of the patient's symptoms, and improvement in the patient's quality of life.

As described above, effective pharmacological treatments exist that can slow progression of the heart failure and extend the patient's life. However, these drugs are rarely used at their therapeutic levels because physicians have no easily accessible method to demonstrate effectiveness of increased doses of the drug. Instead, side effects (which are manageable by careful adjustment in other medications, such as diuretic dose) often result in drugs being used at sub-optimal levels. Further, patients often have poor compliance with their drug therapy. Even for those patients who are able to self-manage their diuretic therapy, weight tracking is an insensitive indicator of increasing volume overload. Hence, detecting an impending event (such as by measuring frequencies of changes in a biomarker level) would allow avoidance of dangerous events by careful adjustment of therapy, diuretics and diet.

Currently, obtaining measurements of heart conditions require access to costly equipment, expert knowledge, and for the patient, a visit to the hospital or clinic. Performing regular visits for serial assessment of the patient's condition is therefore impractical due to limitations such as patient access, long waiting lists, and high cost. Further, these measurements provide information only on the underlying macro-physiology but no specific information on what is happening at the cellular level with respect to neurohormonal control, sympathetic neurotransmitter control, and the process of cardiac remodeling.

However, there is only a weak correlation between signs and symptoms (e.g. shortness of breath, non-specific fatigue and edema) and severity of the heart problem Therefore, the cardiologist relies on infrequent physical measurements often only performed at the time of hospital presentation or during a hospital stay. The generalist physician and healthcare team who deliver routine care to the patient have access to less information on which to make clinical decisions on patient care.

The measurement of blood chemistries (for example, electrolytes, creatinine, hemoglobin, and blood urea nitrogen) is a standard component of the patient's care plan. These are laboratory tests that require a blood specimen to be drawn at the point of care (i.e., in the physician's office, the heart failure clinic, or the hospital). Consequently, laboratory tests are performed relatively infrequently (e.g., every 3 months during a scheduled visit or when the patient is being assessed because of a deteriorating condition). Therefore, these laboratory tests do not predict or detect changes in the patient's condition rapidly enough to prevent an adverse event, such as acute decompensation. Nor are they performed often enough to enable optimal drug titration.

A consequence of sub-optimal control of the patient's condition is a high incidence of hospital admission and readmission. The sequence of events that result in hospitalization often occurs in the home, outside the care setting, away from sophisticated technologies (e.g., echocardiography), laboratory tests, and the expert eye of the caregiver.

There is currently no method to track the occurrence of these events in a patient's home. Failure to detect these events at an early stage results in the onset of deleterious consequences, worsened prognosis, and increased resource utilization.

Consequently, the quality of care available to heart failure patients and their resultant prognosis is lower than would be possible if more objective and predictive measurements were available in the home or remote care-setting to steer the treatment plan.

The biomarkers that can be monitored for excursion from baseline levels, in amplitude and or frequency, can include one or more of the following markers: a marker of inflammation, such as, for example, a soluble adhesion molecule (e.g., E-selectin, P-selectin, intracellular adhesion molecule-1, or vascular cell adhesion molecule-1), Nourin-1, a cytokine (e.g., interleukin-1β, -6, -8, and -10 or tumor necrosis factor-alpha), an acute-phase reactants (e.g., hs-CRP), CRP, meyloperoxidase (MPO) neutrophils, or white blood cell count; a marker of plaque stability, such as, for example, oxidized—low-density lipoprotein (O-LDL) or any other chemical marker which is formed within the plaque and seeps back out into the bloodstream can similarly be used as a predictive marker; a marker of plaque rupture, such as, for example, malondialdehyde-modified LDL (MDA-LDL) or CD40 ligand; a marker of thrombus formation, such as, for example, (fe) thromboxane; a marker of myocardial ischemia, such as, for example, ischemia-modified albumin, oxygen-regulated peptide (ORP150), free fatty acid, Nourin-1, urotensin in all its forms and urotensin-related peptides, and other known markers; a marker of myocardial apoptosis or injury, such as, for example, cardiac troponins, including the isoforms troponin I and troponin T (TnI and TnT, respectively), as well as urotensin in all its forms and urotensin-related peptides; a marker of left ventricular volume overload and myocardial stretch, such as, for example, natriuretic peptides, A-type-(ANP), B-type-(BNP), and C-type-(CNP) natriuretic peptide and their N-terminal prohormones (N-ANP, N-BNP, and N-CNP); a marker of anemia, such as, for example, a hemoglobin level or hematocrit measurement; a marker of renal function, such as, for example, creatinine or Cystatin C; a marker of electrolyte balance, such as, for example, Na⁺ or K⁺ concentrations; or a marker of sodium retention, such as, for example, uroguanylin.

Diagnostic Device

A homecare diagnostic device enables a heart failure patient or an at risk patient and health care provider to safely optimize the care plan, and to track and steer the patient's therapy, response to therapy, diet, and lifestyle. The device can measure and record the levels of one or more biomarkers, record patient input regarding signs and symptoms of disease, provide feedback to the patient, and provide recorded results to a health care provider.

Using the device, the patient's condition can be monitored remotely from a dedicated health care facility, such as doctor's office or hospital. Providing information to optimally manage the patient's condition helps to prevent further heart failure.

The information can also help to predict the onset of cardiovascular events arising from for example, the instability of a plaque or the rupture of an atherosclerotic plaque, thereby allowing early intervention. Use of the device can ensure that interventions can act to stabilize a plaque before rupture or prevent further cardiovascular damage after plaque rupture. The device can further assist the health care provider measure the effectiveness of both pharmacological and non-pharmacological aspects of the care plan, and to monitor the progression of heart failure. The device can also aid in assessing the patient's compliance to therapy, short term risks and future prognosis.

In another embodiment, the device may be used to alert a patient of a longer term risk. For example, fatty acid levels can be used to assess longer term cardiovascular risk. If a user regularly monitors fatty acid levels and determines that they become elevated early enough, lifestyle changes, including diet and exercise, may be sufficient to lower the fatty acid levels and so reduce long term cardiovascular risk.

The device can be used as either a chronic or as an acute monitoring system. As an acute monitoring system, clinical symptoms such as pain, breathlessness, nausea may prompt the use of the device to determine the level of appropriate biomarkers such as markers of ischemia, thrombus formation or cardiovascular stress. In monitoring a chronic disease state the device may be used to identify dangerous trends even when the patient is asymptomatic. For example, frequent cases of silent ischemia resulting from neural damage may cause significant myocardial damage but may go completely undetected. Regular monitoring of appropriate markers will allow detection of rapid concentration excursions of the levels of such markers. By detecting the frequency of such events, peak values, running average concentrations, average time between excursions, the progression of a disease state may be monitored allowing timely medical intervention. As an example, ischemia markers and/or thrombosis markers (such as thromboxane and MDA-LDL) and/or cardiac necrosis markers (such as Troponins or myoglobin) could be used to detect silent infarcts. Peaks in these correlated markers may appear infrequently if the disease is under control. However, as heart failure progresses, peaks in such markers may appear more frequently.

The biomarkers measured by the device can include: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, or a marker of sodium retention. In addition, the device can include probes for measuring the patient's vital signs, such as weight, temperature, heart rate, variability of heart rate, breathing rate, blood pressure, and blood oxygen saturation (measured, for example, by pulse oximetry). The device can record electrical measurements, such as an electrocardiogram, from the patient. The device can present queries to the patient and record the patient's responses. The queries can relate to the patient's condition, such as whether the patient is suffering any symptoms or when medication was taken.

In general, the patient will use the device on a regular basis as instructed by a caregiver. For example, the patient may use the device daily, every other day, weekly, or on any other appropriate interval. Under certain circumstances, fewer than all available tests will be performed. For example, a patient may perform a blood pressure measurement on a daily basis, but measure a biomarker on a weekly basis. Based on the results of the tests, the device can respond with instructions for the patient. The instructions can be configured based on a treatment algorithm. The algorithm can be adjusted to suit the needs of the patient. For example, the health care provider can enter information specific to a particular patient (such as a threshold value for a biomarker) into the device.

The patient may or may not see the actual recorded values of these biomarkers. As long as all biomarkers are within pre-set “normal” limits, the display may simply display “NORMAL” or “STABLE” or any other appropriate icon. Outside these limits the display may alert the patient with an icon such as “CHECK_UP” or “URGENT” or any other appropriate icon. The data could be automatically sent to the physician who further evaluate the patient if critical values are exceeded. These values may be actual levels, rate of change, or frequency of excursion events.

By use of an embedded algorithm, the device could also be used to steer therapy. For example, the instrument may contain an algorithm with certain parameters pre-set by a health care professional. In this way the patient would be able to adjust his own medication based on monitor readings. Once again, it may or may not be desirable for the patient to see an actual clinical value. The calculation could be encoded within the device to advise a particular medication regime based on current and historical data stored within the instrument.

The biomarkers can be measured in a sample. The sample is taken from the patient and can be a sample of blood, plasma, serum, saliva or urine. In one embodiment, the sample is a blood sample. Such a sample may be taken by the patient by, for example, collecting a blood sample having a volume of less than one microliter up to a volume of several hundred microliters following puncture of the skin with an appropriate lancing device. The biomarkers monitored can be detected using, for example, an immunoassay, a biosensor, an ion-selective electrode, or another suitable technology.

For example, the markers can be detected using an immunoassay. An immunoassay is performed by contacting a sample from a subject to be tested with an appropriate antibody under conditions such that immunospecific binding can occur if the marker is present, and detecting or measuring the amount of any immunospecific binding by the antibody. Any suitable immunoassay can be used, including, without limitation, competitive and non-competitive immunoassay systems or ligand-binding systems known to one skilled in the art.

For example, a marker can be detected in a fluid sample by means of a one-step sandwich assay. A capture reagent (e.g., an anti-marker antibody) is used to capture the marker. Simultaneously, a directly or indirectly labeled detection reagent is used to detect the captured marker. In one embodiment, the detection reagent is an antibody. Such an immunoassay or another design known to one skilled in the art can be used to measure the level of an aforementioned biomarker in an appropriate body fluid.

A GFR marker (e.g. serum creatinine) can be measured using a biosensor, an enzymatic assay, or amperometrically. See, for example, Erlenkotter A, Anal Bioanal Chem. 2002 January; 372(2):284-92; Leger F, Eur J Cancer. 2002 January; 38(1):52-6; and Tombach B, Clin Chim Acta. 2001 October; 312(1-2):129-34, each of which is incorporated by reference in its entirety.

The measurement of a biomarker by both immunoassay and biosensor (e.g. calorimetrically) has been demonstrated by Metrika with their patented MODM™ (Micro Optical Detection Method) technology. This integrates miniaturized digital electronics, micro-optics and solid-state chemistries into an easy to use, low-cost, single-use instrument. MODM technology is designed for simultaneous measurement of immunodiagnostic and general chemistries in less than ten minutes. Ostex International Inc. has used the same technology to develop the OSTEOMARK NTx Point-of-Care (POC). This is a disposable single use device that provides a normalized measurement of the bone marker ‘NTx’ by measuring NTx and creatinine levels in a sample and then calculating the ratio result. The POC is intended for use in a physician's office and takes 5 minutes to process.

The device can be included in a diagnostic kit, which can optionally include one or more of the following: instructions for using the kit for event detection, diagnosis, prognosis, screening, therapeutic monitoring or any combination of these applications for the management of patients with pre-heart failure, heart failure, or hypertension; a disposable testing cartridge containing the necessary reagents to conduct a test; or an instrument or device that measures the result of biomarker testing and optionally, allows manual or automatic input of other parameters, storage of said parameters, and evaluation of said parameters alongside or separate from the evaluation of the measured biomarkers.

The testing cartridge or cartridges supplied in the kit allows the user to measure at a minimum, a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, or a marker of sodium retention.

In one embodiment, the testing cartridge or testing cartridges allow the sequential or serial measurement of a marker of inflammation, a marker of plaque instability and/or a marker of plaque rupture.

The testing cartridge or testing cartridges allow the sequential or serial measurement of a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, or a marker of sodium retention. A combination cartridge can test two or more different markers from a single sample.

The instrument (durable or disposable), at a minimum, measures the result of biomarker testing and optionally, allows manual or automatic input of other parameters, storage of said parameters, and evaluation of said parameters with or separate to the measured biomarkers.

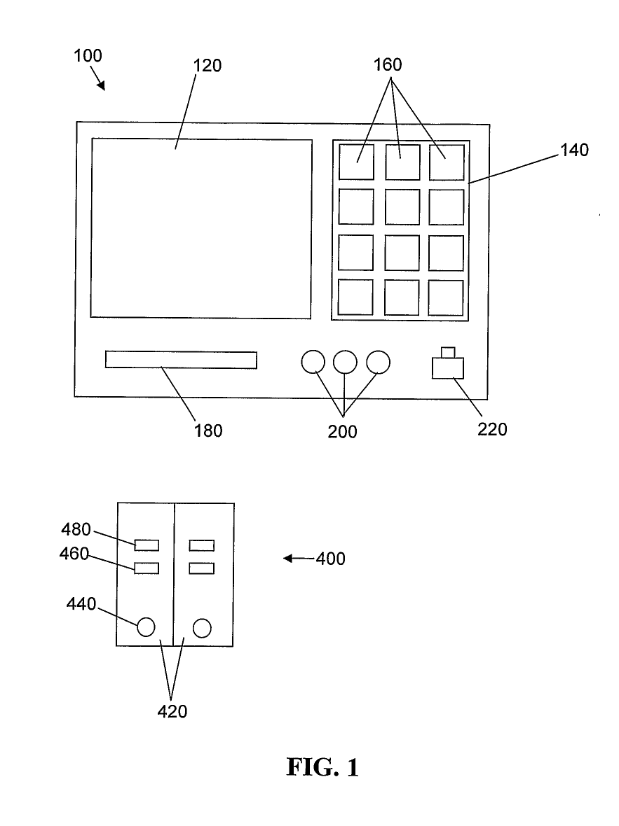

Referring to FIG. 1, diagnostic device 100 includes display 120 and input region 140. The display 120 may be used to display images in various formats, for example, joint photographic experts group (JPEG) format, tagged image file format (TIFF), graphics interchange format (GIF), or bitmap. Display 120 can also be used to display text messages, help messages, instructions, queries, test results, and various information to patients. In some implementations, display 120 supports the hypertext markup language (HTML) format such that displayed text may include hyperlinks to additional information, images, or formatted text. Display 120 can further provide a mechanism for displaying videos stored, for example in the moving picture experts group (MPEG) format, Apple's QuickTime format, or DVD format. Display 120 can additionally include an audio source (e.g., a speaker) to produce audible instructions, sounds, music, and the like.

Input region 140 can include keys 160. In one embodiment, input region 140 can be implemented as symbols displayed on the display 120, for example when display 120 is a touch-sensitive screen. Patient instructions and queries are presented to the patient on display 120. The patient can respond to the queries via the input region.

Device 100 also includes cartridge reader 180, which accepts diagnostic test cartridges for reading. The cartridge reader 180 measures the level of a biomarker based on, for example, the magnitude of a color change that occurs on a test cartridge 400. Device 100 also includes probe connections 200, which connect probes (e.g., a probe of weight, temperature, heart rate, variability of heart rate, breathing rate, blood pressure, or blood oxygen saturation) to the device.

Device 100 further includes a communication port 220. Communication port 220 can be, for example, a connection to a telephone line or computer network. Device 100 can communicate the results of patient tests to a health care provider from a remote location. Likewise, the health care provider can communicate with the device 100 (e.g., to access stored test results, to adjust device parameters, or send a message to the patient).

Cartridge 400 is shown with two testing zones 420. In general, a cartridge can include 1, 2, 3, 4, or 5 or more testing zones. Each testing zone 420 can test the level of a biomarker. Each testing zone 420 includes a sample input 440, a control result window 460 and a test result window 480. In one embodiment, the cartridge 400 is an immunochromatographic test cartridge. Examples of immunochromatographic tests and test result readers can be found in, for example, U.S. Pat. Nos. 5,504,013; 5,622,871; 6,235,241; and 6,399,398, each of which is incorporated by reference in its entirety.

A patient can use device 100 for testing and recording the levels of various biomarkers that provide information about the patient's health. Various implementations of diagnostic device 100 may access programs and/or data stored on a storage medium (e.g., video cassette recorder (VCR) tape or digital video disc (DVD); compact disc (CD); or floppy disk). Additionally, various implementations may access programs and/or data accessed stored on another computer system through a communication medium including a direct cable connection, a computer network, a wireless network, a satellite network, or the like.

The software controlling the diagnostic device and providing patient feedback can be in the form of a software application running on any processing device, such as, a general-purpose computing device, a personal digital assistant (PDA), a special-purpose computing device, a laptop computer, a handheld computer, or a network appliance.

A diagnostic device may be implemented using a hardware configuration including a processor, one or more input devices, one or more output devices, a computer-readable medium, and a computer memory device. The processor may be implemented using any computer processing device, such as, a general-purpose microprocessor or an application-specific integrated circuit (ASIC). The processor can be integrated with input/output (I/O) devices to provide a mechanism to receive sensor data and/or input data and to provide a mechanism to display or otherwise output queries and results to a service technician. Input device may include, for example, one or more of the following: a mouse, a keyboard, a touch-screen display, a button, a sensor, and a counter.

The display 120 may be implemented using any output technology, including a liquid crystal display (LCD), a television, a printer, and a light emitting diode (LED). The computer-readable medium provides a mechanism for storing programs and data either on a fixed or removable medium. The computer-readable medium may be implemented using a conventional computer hard drive, or other removable medium such as those described above with reference to. Finally, the system uses a computer memory device, such as a random access memory (RAM), to assist in operating the diagnostic device.

Implementations of a diagnostic device can include software that directs the patient in using the device, stores the result of biomarker measurements, determines whether a tested biomarker level requires medical attention for the patient, instructs the patient in adjusting or maintaining therapy, and communicates the patient's information to his or her caregiver. At risk patients or patients suffering from, for example, heart failure, can use the device.

The device 100 can provide access to applications such as a medical records database or other systems used in the care of patients. In one example, the device connects to a medical records database via communication port 220. Device 100 may also have the ability to go online, integrating existing databases and linking other websites. Online access may also provide remote, online access by patients to medical information, and by caregivers to up-to-date test results reflecting the health of patients.

The device can be used in the hospital, physician's office, clinic, or patient's home either by the patient or an attendant care giver. In one embodiment, the invention is practiced in the patient's home allowing the patient to be monitored, his or her therapy optimized, and adverse events that require hospitalization to be avoided.

The device can provide information on the patient's status and provide instructions or other actionable information to the healthcare professional and/or the patient. Examples, without limitation, of instructions that can be given include: contact caregiver, no change in care plan necessary, change fluid intake, withhold potassium supplementation, increase potassium supplementation, change diuretic dose, withhold diuretic, introduce another diuretic. The objective is to track the patient's condition and steer him or her toward a stable condition through appropriate interventions made by the patient or the caregiver. Algorithms for treatment decisions are known. An example of a set of treatment algorithms can be found in: Healthcare Guideline; Congestive Heart Failure in Adults, Institute for Clinical Systems Improvement, Release July 2003; and Silver M, Pisano C, Cianci P, Outpatient management of heart failure: Program development and experience in clinical practice, Advocate Christ Medical Center, Oak Lawn, Ill., Post Graduate Institute for Medicine 2003, each of which is incorporated by reference in its entirety.

Decision Points

The device can be configured to respond to the measured level of a biomarker, in particular when the increased frequency and amplitude of changes in the normal levels of the biomarker indicates a change in the patient's health status. For example, the device can be configured to store the results of tests and determine the frequency and amplitude of changes in the levels of markers over time. Such changes in results over time can be an acute change or a chronic change. An acute change can be a significant change in the level of a biomarker over a short period of time. The magnitude of change and period of time can be different for each biomarker. The device can be configured to compare each new test result either to a stored values of recent test results (e.g., the previous 1, 2, 3, 4, 5 or more results), or to an aggregate measure of recent test results (such as an average) to determine if an acute change has occurred. In one example, an acute change is detected by the percentage change in a test result from the previous result.

Chronic changes can be detected as well. A chronic change can be a change in the level of a biomarker that occurs over a long period of time. For example, a chronic change can occur such that many testing intervals pass without an acute change being detected, yet the level of biomarker is significantly different. To detect a chronic change, the device can compare the results of each new test to a stored result of an earlier test, or to an aggregate measure of earlier tests. For detecting chronic changes, the earlier test can be, for example, 4-12 weeks prior to the new test result. In one example, the aggregate measure can be a rolling average, such as a 4-week, 8-week, or 12-week rolling average.

The device can also be configured to compare test results to a stored threshold value or range. The threshold value can be an upper or lower limit or range of values. Thus, the device can determine if the measured value of a marker, or group of markers, is a safe level, a dangerous level, or indicates an emergency. The device can alert the patient to the results of the test and can be configured, when appropriate to instruct the patient to seek medical care.

The device can also be configured to track combinations of markers, for example, an average value of two markers, the difference in level between two markers, a ratio of the levels of two markers, or whether two or more markers exceed their respective threshold values at the same time. The device can be configured to track one or more markers in combination with a patient's signs and symptoms.

The device can be personalized for a patient. The threshold values and other parameters for each biomarker can be adjusted (for example, by a physician or other caregiver) based on the circumstances of the patient, such as, for example, age, gender, or disease status or risk for developing heart failure. The questions and responses that the device presents to the patient can also be adjusted.

Examples of how the device can record, detect changes, and respond to detected changes in the level of a biomarker are presented below. The threshold values and levels of biomarkers referred to below are not limiting, may not be appropriate for all patients, and are for purposes of example only.

Marker of Left Ventricular Volume Overload and Myocardial Stretch

In one embodiment, the device is configured to measure the biomarker BNP in a patient sample. The device can track the patient's BNP level as a function of time and detect changes in the BNP level. The changes can be acute or chronic. When a change in BNP level is detected, the device can respond with a request for additional input for the patient or instructions for the patient.

The device can determine a patient's baseline level of BNP, against which future measurements of BNP will be compared. The baseline level can be set based on data on the influence of the patient's gender, age, body mass, and degree of hypertrophy. The baseline can also be refined to set reasonable treatment targets for a patient taking into consideration the degree of disease comorbidities and the patient's prognosis. A series of BNP measurements can be used to set a baseline for a patient.

For example, the baseline can be defined as the average of the most recent two test results with an increase of maximum 10% (compared to the previous baseline) out of the last four tests. The following test results are excluded from the calculation:
Any test result flagged with an acute symptom
Any test of the 2 used for the calculation is older than 28 days
The last 4 tests have been done in less than 4 days

Under certain conditions, no baseline value will be available, such as the first use of the device (i.e., no test results have been recorded); after the device has been reset; or when any of the test results used for the baseline calculation is older than 28 days. By testing 4 times over at least 4 days, the initial baseline can be calculated. When the baseline is defined in this way, the device cannot give warnings for acute deterioration over this initial 4 day period. In case one value of the two used for the calculation is older than 28 days, one additional test can be sufficient to calculate the baseline. The baseline can be a variable baseline, changing as the most recent test results change in value.

The device can detect acute changes in BNP level, and advise the patient to take appropriate responses. Criteria for determining an appropriate response can include the patient's initial BNP level, which can reflect the patient's risk profile; the percentage change in BNP level; the presence or absence of acute symptoms; and the evolution of BNP values to confirm a trend and exclude assay-to-assay, physiological, and statistical variations. A device may instruct the patient to begin monitoring additional biomarkers such as cardiac structural makers such as cTnT to ascertain that cardiac cell death is not escalating and that the disease is contained.

When an acute increase in BNP level is detected by the device, the device can query the patient for the presence of acute symptoms. In advising a patient of a response to take to increased BNP levels, the presence of one or more acute symptoms can be a deciding factor. Acute symptoms can include chest pain (AMI); a squeezing or crushing chest feel (AMI); pain radiating to neck, left arm (AMI); sweating, nausea, or vomiting (AMI, Stroke, pulmonary TE); loss of consciousness; acute dyspnea (AMI, decompensation, pulmonary thrombo-embolism); palpitations without exercise; dyspnea when laying down (right heart decompensation); sudden headache (stroke); and sudden vision impairment (stroke). See, for example, Harrisson T R. et al., Principles of Internal Medicine. McGraw Hill, Inc. 1983, 1432-34 & 1353-58 & 2038-39, which is incorporated by reference in its entirety. When the patient indicates that any acute symptoms are present, the device can advise the patient to seek medical care at once.

If there is an acute increase in BNP level, but the patient is not experiencing any acute symptoms, the device's response can depend on the percentage change in BNP level and the absolute BNP level. In general, a large percentage increase in BNP level and a high absolute level can indicate a deterioration in the patient's condition, and the device can respond by prompting the patient to seek medical care at once. A smaller percentage change and lower absolute level may not require immediate medical attention, and the change in BNP level can be confirmed by a second test. In one example, the severity of a patient's disease can be stratified by absolute BNP levels as follows (see, for example, Clerico A, et al., Clin Chem Lab Med 2002 April; 40(4): 371-7; and Nomura H, et al., J Am Geriatr Soc 2002 September; 50(9): 1504-9, each of which is incorporated by reference in its entirety):

| | | |
|---|---|---|
| 20 | pg/mL | Healthy |
| 20-50 | pg/mL | 1 risk factor: hypertension or age |
| 50-100 | pg/mL | 2 risk factors: hypertension, age, post-AMI |
| >100 | pg/mL | chronic hart failure patient NYHA classes 1-4 |

Changes in BNP level can also be grouped by severity, for example, no increase, an increase of less than 10%, 10-20%, 20-30%, 30-40%, or 40% or more.

A second test can exclude assay-to-assay or physiological variations and thus confirm the increase. The second test can be given after a predetermined interval, which can vary depending on the severity of the increase (e.g., within 30 minutes, 60 minutes, the same day, or within 24 hours of the first test). If the second test result is a lower BNP value, then a third test can be performed. The third test can confirm an increase in this case, or, for example, exclude a non-pathological transient rise of more than 20% due to exercise.

For example, if the BNP level increases by 10% or less (and the patient has no acute symptoms), the device can prompt the patient to perform a second test. The second test can be performed the next day (for example, if the patient's BNP level is less than 50 pg/mL) or sooner, such as thirty minutes later (for example, if the patient's BNP level is 50 pg/mL or greater). If the BNP level has increased by 10-20%, the device can prompt the patient to perform a second test, for example, within thirty minutes of the first test. If the BNP level has increased by more than 20%, the device can prompt the patient to seek medical care at once. An increase of more than 30% can be regarded as the strongly indicative for ischemia and AMI or an acute heart decompensation. See, for example, Kyriakides Z S et al., Clin Cardiol 2000 April; 23(4): 285-8; and Nakamura T, et al., J. Am. Coll. Cardiol. 2002 May 15; 39 (10): 1657-63, each of which is incorporated by reference in its entirety. If the patient's BNP level has not increased, or increased by less than 5%, the device can prompt the patient to perform a second test at a predetermined interval, such as seven days.

The device can respond to the results of the second test. If the second test is performed on the day after the previous test (e.g., when the patient's BNP level is less than 50 pg/mL), the device can respond as follows. If the second test reveals a BNP level more than 20% above the baseline, the patient is instructed to seek medical care at once. The patient can be instructed to perform a third test if the second test reveals a BNP level that is between 0 and 20% higher than the baseline. The third test can be performed, for example, on the day following the second test. If the third test indicates that the patient's BNP level is between 10% and 20% higher than the baseline, the patient is instructed to seek medical care at once. However, if the third test reveals a BNP level between 0 and 10% higher than the baseline, the baseline can be adjusted to the average of the previous baseline and the result of the third test. The patient is instructed to resume a regular test schedule, such as once a week.

If the second test is performed within thirty minutes of the previous test (e.g., when the patient's BNP level is 50 pg/mL or greater), the device can respond as follows. When the second test result is 20% or more above the baseline, the patient is instructed to seek medical care at once. The patient can be instructed to perform a third test if the second test reveals a BNP level that is between 0 and 20% higher than the baseline. The third test can be performed within thirty minutes of the second test (such as when the second test result was between 10% and 20% above the baseline) or within four hours of the second test (such as when the second test result was between 0 and 10% above the baseline). If the third test indicates that the patient's BNP level is between 10% and 20% higher than the baseline, the is instructed to seek medical care at once. However, if the third test reveals a BNP level between 0 and 10% higher than the baseline, the baseline can be adjusted to the average of the previous baseline and the result of the third test. The patient is instructed to resume a regular test schedule, such as once a week.

Chronic Changes

The device can detect chronic changes in BNP level; in other words, slow changes that accumulate over time to reflect a change in the patient's condition. A chronic change can be measured, for example, by observing changes in a rolling average of BNP values, such as a rolling 2-week average. To exclude increases of an acute nature, or due to a temporary event (such as exercise), only those chronic increases that are manifested for at least two weeks where the increases outnumber the decreases can be considered as chronic increases. A chronic increase can be small (e.g., approximately 10%) when consistent over a long time (such as one month) or can be large (for example, approximately 20%) over a relatively short term (such as two weeks). It can be important to exclude increases due to assay-to-assay variability, physiological rises, etc., before concluding that a chronic increase has occurred. In order to due so, sufficient test results have to be available. Therefore, upon suspicion of a chronic increase, patients can be instructed to perform more tests.

A rolling average can be the average of test results performed within a given time frame. For example, a rolling two week average can be the average of results recorded over the previous 15 days, a rolling 4 week average can be the average of results recorded over the previous 29 days, and a rolling 12 week average can be the average of results recorded over the previous 85 days. When calculating a rolling average, any test result recorded with an acute symptom flag (i.e., a test result where the patient was suffering an acute symptom at the time of the test) can be excluded. Under certain circumstances, a rolling average cannot be calculated, such as the first use of the system, following a system reset, or when the system has not been used over the relevant length of time (e.g., 2, 4 or 12 weeks). By testing once a week over at least 15 days (three test results), an initial 2-week rolling average can be calculated. This means that the device cannot give warnings for chronic deterioration over this initial 2-week period.

When a chronic increase in BNP levels is detected, the device can query the patient for the presence of chronic symptoms. Examples of chronic symptoms include increasing fatigue in general (heart performance reduction); shortening of walking distance or step climbing (heart performance reduction); aggravating chronic dyspnea (right heart decompensation, multiple pulmonary thrombo-embolism); palpitations without exercise; aggravating dyspnea when laying down (decompensation); aggravating swollen feet or legs; or memory loss or paralysis or equilibrium disturbance. When the patient indicates that any chronic symptoms are present, the device can advise the patient to seek medical care at once.

If there is a chronic increase in BNP level, but the patient is not experiencing any chronic symptoms, the device's response can depend on the percentage change in BNP level and the absolute BNP level. In general, a large percentage increase in BNP level and a high absolute level can indicate a deterioration in the patient's condition, and the device can respond by prompting the patient to seek medical care at once. A smaller percentage change and lower absolute level may not require immediate medical attention, and the change in BNP level can be confirmed by a second test. In one example, the severity of a patient's disease can be stratified by absolute BNP levels as follows:

| | | |
|---|---|---|
| <20 | pg/mL | Healthy |
| 20-50 | pg/mL | 1 risk factor: hypertension or age |
| 50-100 | pg/mL | 2 risk factors: hypertension, age, post-AMI |
| >100 | pg/mL | chronic heart failure patient NYHA classes 1-4 |

Chronic changes in BNP level can also be grouped by the duration in the change, for example, a change in the two-week rolling average, a change in a 4-week rolling average, or a change over a longer interval, such as a change in the 12-week rolling average. In each of these time periods, the changes in BNP level can be grouped by severity, such as no increase, an increase of greater or less than 7.5%, an increase of greater or less than 15%, an increase of less than 10%, an increase of 10-30%, an increase of 30-50%, or an increase of more than 50%.

For example, when the device detects a small, chronic increase in the two-week rolling average (e.g., an increase of less than 10%) in the patient's BNP level, and the patient reports no chronic symptoms, the device can instruct the patient to perform a second test after a predetermined interval, such as 7 days. The device may also instruct the patient to monitor other biomarkers such as cardiac structural makers which may include cTnT, to ascertain that cardiac cell death is not escalating and that the disease is contained. If there is a moderate increase in the patient's two-week rolling average BNP level (e.g., an increase of 10-30%), the device can instruct the patient to perform a second test after a predetermined interval, such as within 24 or 48 hours. A large increase (e.g., of 30-50%) and a small absolute BNP level (e.g., less than 50 pg/mL) can cause the device to instruct the patient to perform a second test after a predetermined interval, such as within 24 or 48 hours. When the device detects a severe increase (e.g., of more than 50%) in the two-week rolling average, it can instruct the patient to seek medical care at once.

If the second test result is a BNP level higher than the previous two-week rolling average, the device can instruct the patient to seek medical care at once. If, on the other hand, the second test result is lower than the previous result, the device can instruct the patient to perform additional test (e.g., one test each day) until the BNP level either returns to its previous level, or the BNP level increases, which will result in a prompt to the patient to seek medical care at once. If the BNP level does not return to its previous level or increase within one week, the device can prompt the patient to seek medical care at once.

When there is a small increase in the 4-week rolling average (e.g., an increase of less than 15%), and the patient reports no chronic symptoms, the device can instruct the patient to perform a second test after a predetermined interval, such as 7 days. When there is a large increase in the 4-week rolling average (e.g., an increase of 15% or greater), the device can instruct the patient to report to his or her health care provider.

When there is a small increase in the 12-week rolling average (e.g., an increase of less than 7.5%), and the patient reports no chronic symptoms, the device can instruct the patient to perform a second test after a predetermined interval, such as 7 days. When there is a large increase in the 4-week rolling average (e.g., an increase of 7.5% or greater), the device can instruct the patient to report to his or her health care provider.

The parameters used by the devices (i.e., the values of percentage change in BNP level, absolute BNP level, patient messages, etc.), can be altered. For example, a physician or other health care provider can adjust the value of acute increase in BNP level required to prompt the patient to seek medical care to a desired value. In this way, the behavior of the device can be tailored according to the preferences of a physician or to the needs of a particular patient or group of patients.

Markers of Renal Function

The following uses, by example, the biomarker creatinine. The accepted method for use in routine care is a measure of creatinine using adjustment with the Cockroft and Gault equation. Creatinine can provide important information on volume status and should be followed in patients during optimization of pharmacological agents (e.g. an ACE inhibitor) and ideally throughout the patient's care. Tests are performed by the patient or the healthcare provider every day, at a suitable testing interval, or with the onset of certain signs and symptoms. An increase in serum creatinine of 0.05 to 0.5 mg/dL is an indication for reassessment of volume status. Renal function declines with age; many elderly patients have a glomerular filtration rate below 50 mL/minute. Further, as stated, an early increase of <30% in the concentration of creatinine is expected when a patient is administered an ACE inhibitor. GFR monitoring using, for example, creatinine is important in these patients.

Further, when using BNP to guide the optimization of pharmacological treatment, an estimate of GFR is essential to avoid under-hydration. An ‘action level’ (e.g. a level that defines a significant reduction in renal perfusion) for GFR will require the healthcare professional and/or the patient to follow a predefined intervention dependent on the rate of change of GFR over time and the absolute level. An intervention might include a change in diuretic dose, withhold the diuretic, introduce another diuretic, change fluid intake, withhold potassium supplementation, increase potassium supplementation, contact the healthcare professional, refer to the Emergency Department, etc). As described, alternative markers of GFR can be used, such as Cystatin C.

Renal function can also be used as a prognostic marker, to provide information on a patient's health over a long period of time. The prognostic value of a measure of renal function (e.g., GFR as determined by a creatinine or Cystatin C measurement) can be independent of the use of renal function to monitor hydration on a short-term basis (e.g., during diuretic use). An average measure of renal function determined over a period of time can be used for prognostic purposes. See, for example, Koenig W, et al., Clin Chem. 10.1373/clinchem.2004.041889 2004 November; and Gottlieb S S, et al., J Card Fail. 2002 June; 8(3):136-41, each of which is incorporated by reference in its entirety.

Marker of Myocardial Apoptosis or Injury

Measurement of a marker of myocardial apoptosis or injury, such as a troponin, in a remote setting using frequent testing is clinically useful. Tests are performed by the patient or the healthcare provider every day, at a suitable testing interval, or with the onset of certain signs and symptoms.

Marker of Inflammation

Measurement of a marker of inflammation in a remote setting is clinically useful. The marker of inflammation can include, for example, E-selectin, P-selectin, intracellular adhesion molecule-1, vascular cell adhesion molecule-1, Nourin-1, interleukin-1β, interleukin-6, interleukin-8, interleukin-10, tumor necrosis factor-alpha, hs-CRP, neutrophils, or white blood cell count. Tests are performed by the patient or the healthcare provider every day, at a suitable testing interval, or with the onset of certain signs and symptoms.

Marker of Anemia

Measurement of a marker of anemia, such as hemoglobin or hematocrit, in a remote setting using frequent testing is clinically useful. Tests are performed by the patient or the healthcare provider every day, at a suitable testing interval, or with the onset of certain signs and symptoms.

Markers of Myocardial Ischemia

Chronic myocardial ischemia is the leading cause of impaired myocardial contractility and heart failure. Ischemia markers (e.g. ischemia modified albumin, oxygen-regulated peptide, and free fatty acid) have a faster release profile than early necrosis markers (e.g. myoglobin or fatty acid binding protein (H-FABP)). Tests are performed by the patient or the healthcare provider every day, at a suitable testing interval, or with the onset of certain signs and symptoms.

Markers of Electrolyte Balance and Markers of Sodium Retention

Levels of electrolyte balance and sodium retention may be determined by measurement of the levels of sodium and potassium ion concentrations in serum. This may be conveniently done using ion selective electrodes.

Other embodiments are within the scope of the following claims.

What is claimed is:

1. A device for predicting heart failure comprising a detector configured to monitor concentration excursions, in samples taken from a patient at regular intervals, a level of a first biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

2. The device of claim 1, further comprising detecting the frequency of concentration excursions from average concentrations of the first biomarker.

3. The device of claim 1, further comprising a display configured to provide an output to the patient.

4. The device of claim 1, wherein the detector is configured to monitor concentration excursions of the level of a second biomarker.

5. The device of claim 1, wherein the detector is configured to monitor concentration excursions of the level of a third biomarker.

6. The device of claim 4, wherein the second biomarker is selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

7. The device of claim 5, wherein the third biomarker is selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

8. The device of claim 1, wherein the first biomarker includes a marker of plaque instability.

9. The device of claim 6, wherein the first biomarker includes a marker of inflammation and the second biomarker is a marker of plaque instability.

10. The device of claim 7, wherein the first biomarker includes a marker of inflammation, the second biomarker is a marker of plaque instability, and the third biomarker is a marker of plaque rupture.

11. The device of claim 1, wherein the marker of inflammation includes E-selectin, P-selectin, intracellular adhesion molecule-1, vascular cell adhesion molecule-1, Nourin-1, interleukin-1β, interleukin-6, interleukin-8, interleukin-10, tumor necrosis factor-alpha, hs-CRP, myeloperoxidase, neutrophils, or white blood cell count.

12. The device of claim 1, wherein the marker of plaque instability includes oxidized-LDL.

13. The device of claim 1, wherein the marker of thrombus formation includes (fe) thromboxane.

14. The device of claim 1, wherein the marker of plaque rupture includes malondialdehyde-modified LDL (MDA-LDL).

15. The device of claim 1, wherein the marker of myocardial apoptosis or injury includes cardiac troponin I, troponin T, myoglobin, creatine kinase or creatine kinase MB (CK MB), urotensin, or urotensin-related peptide.

16. The device of claim 1, wherein the marker of myocardial ischemia includes ischemia-modified albumin, oxygen-regulated peptide (ORP150), free fatty acid, Nourin-1, urotensin, or urotensin-related peptide.

17. The device of claim 1, wherein the marker of anemia includes hemoglobin or hematocrit.

18. The device of claim 1, wherein the marker of renal function includes creatinine or Cystatin C.

19. The device of claim 1, wherein the marker of electrolyte balance includes Na⁺ or K⁺.

20. The device of claim 1, wherein the marker of sodium retention includes uroguanylin.

21. The device of claim 1, further comprising a probe for measuring a vital sign of the patient.

22. The device of claim 21, wherein the probe is capable of measuring a weight, a heart rate, variability of heart rate, a breathing rate, a blood pressure, a temperature, a blood oxygen saturation, or an electrocardiogram of the patient.

23. The device of claim 1, further comprising a memory capable of storing the results of regular measurements of the level of the first biomarker.

24. The device of claim 23, wherein the device is configured to compare the result of a measurement of the level of the first biomarker to stored results from previous measurements.

25. The device of claim 24, wherein the memory is further capable of storing a threshold value of the level of the first biomarker.

26. The device of claim 25, wherein the device is configured to compare the result of a measurement of the level of the first biomarker to the threshold value and to previous measurements.

27. The device of claim 26, wherein the device is configured to instruct the patient to contact his physician when the device detects concentration excursions in the levels of the first biomarker.

28. The device of claim 26, wherein the device is configured to instruct the patient to alter a treatment plan when the device detects concentration excursions in the levels of the first biomarker occurring at high frequency.

29. The device of claim 26, wherein the device is configured to further instruct the patient to obtain a measurement of a second biomarker and/or third biomarker.

30. The device of claim 1, further comprising a display for displaying the results of the measurement, a patient query, or a patient instruction.

31. The device of claim 30, further comprising an input device for supplying a response to a patient query.

32. The device of claim 30, wherein the device is configured to provide a personalized patient instruction in response to the results of the measurement.

33. The device of claim 1, further comprising a communication port configured to transmit a result of a measurement to a recipient.

34. The device of claim 33, wherein the communication port is further configured to receive information from the recipient.

35. A method of monitoring a patient for heart failure comprising measuring and detecting concentration excursions, in samples taken from a patient at regular intervals, the levels of a first biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

36. The method of claim 35, further comprising providing an output to the patient.

37. The method of claim 35, further comprising comparing the measured level of the first biomarker to a threshold value and to previous measurements.

38. The method of claim 35, further comprising instructing the patient to obtain a measurement of a second biomarker and/or a third biomarker when rapid concentration excursions are detected in the levels of the first biomarker.

39. The method of claim 35, further comprising measuring and detecting concentration excursions in a sample taken from a patient, a level of a second biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

40. The method of claim 39, further comprising measuring and detecting concentration excursions in a sample taken from a patient, a level of a third biomarker selected from the group consisting of: a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

41. The method of claim 35, further comprising determining whether the patient is suffering from one or more symptoms associated with heart failure.

42. The method of claim 35, further comprising measuring a weight, a heart rate, variability of heart rate, a breathing rate, a blood pressure, a temperature, a blood oxygen saturation, or an electrocardiogram of the patient.

43. A health care kit comprising:
a test cartridge including a sample port and a first assay, wherein the first assay recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention; and
a device including a detector configured to measure and to monitor concentration excursions of a level of the biomarker recognized by the assay.

44. The kit of claim 43, wherein the device is configured to provide an output to a patient.

45. The kit of claim 43, wherein the first assay includes an antibody that recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

46. The kit of claim 43, further comprising a second test cartridge including a sample port and a second assay, wherein the second assay recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

47. The kit of claim 46, wherein the second assay includes an antibody that recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

48. The kit of claim 46, further comprising a third test cartridge including a sample port and a third assay, wherein the second assay recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

49. The kit of claim 48, wherein the third assay includes an antibody that recognizes a marker of inflammation, a marker of plaque stability, a marker of thrombus formation, a marker of plaque rupture, a marker of myocardial ischemia, a marker of myocardial apoptosis or injury, a marker of left ventricular volume overload or myocardial stretch, a marker of anemia, a marker of renal function, a marker of electrolyte balance, and a marker of sodium retention.

50. The kit of claim 43, wherein the test cartridge includes a second assay, the second assay being different from the first assay.
